(19) United States
(12) Patent Application Publication
(10) Pub. No.: US 2008/0015591 A1
(43) Pub. Date: Jan. 17, 2008

(54) Threaded Guide for an Orthopedic Fixation Plate

(76) Inventors: Javier E. Castaneda, Miami, FL (US); Chris Bremer, Warsaw, IN (US); Steve Donnelly, Willoughby, OH (US); Pam Guzman, Wayne, IN (US); Dennis Stoller, Wayne, IN (US)

Correspondence Address: GORDON & JACOBSON, P.C., 60 LONG RIDGE ROAD, SUITE 407, STAMFORD, CT, 06902, US

(21) Appl. No.: 11/457,305

(22) Filed: Jul. 13, 2006

(51) Int. Cl. A61F 2/30

(52) U.S. Cl. 606/69

(57) ABSTRACT

A short guide for temporary fixation to a fastener hole in a bone plate, includes a bottom threaded portion, a top portion, and a central passage for guiding an orthopedic device. The threaded end is preferably provided with a triple lead taper thread. The central passage is sized for receiving an orthopedic device, e.g., a drill bit or K-wire. The top portion is provided with tool engagement structure for coupling to a guide driver. The length of the device permits multiple guides to be pre-assembled to the plate without obscuring and crowding the area of interest. When the bone plate includes polyaxial bushings, the guides are initially lightly screwed therein, permitting the surgeon to modify the angle of each guide and bushing to a selected orientation before locking the bushing at an orientation by tightening the guide into the bushing.

### CROSS-REFERENCE TO RELATED APPLICATIONS

This application is related to U.S. Ser. No. 11/011,917, filed Dec. 14, 2004 and U.S. Pub. No. 2005/0049594A1 to Wack et al., the contents of which are hereby incorporated by reference herein in their entireties.

### BACKGROUND OF THE INVENTION

1. Field of the Invention

This invention relates broadly to surgery and, more specifically, to orthopedic fixation plate systems. More particularly, this invention relates to guides which are removably couplable to orthopedic fixation plates.

2. State of the Art

Orthopedic surgery to repair fractures is challenging. Proximal humoral fractures are no different in that regard. As the fracture site is prepared to receive a bone plate, the surgeon must handle numerous instruments and then properly locate the implant in the desired fixation location and temporally secure it for evaluation before permanently securing the implant to bone. In placing the implant, there is no defined manner of handling the plate securely while manipulating it among the skin, fat, and muscle of the patient to its desired location easily. Furthermore, the available plates include limited locations for placement of K-wires which are used for temporarily securing the implant against the bone. Often, there is a desirable location for temporarily anchoring the plate but such location cannot be used due to the location of a screw hole of unsuitable dimension for the K-wire.

In addition, after evaluative placement when it is necessary to permanently secure the plate with screws, the surgeon may encounter difficulty using the currently available drill guides to drill holes for the screw. For example, U.S. Pat. No. 5,954,722 to Bono and U.S. Pub. No. 2005/0049594A1 to Wack et al., previously incorporated by reference in their entireties, include polyaxial bushings provided in holes of a plate. The systems are intended for use with an approximately six inch long drill guide that may be screwed into a bushing and which allows the surgeon to drill a hole in the bone along the polyaxial bushing axis without injuring surrounding tissue. Unfortunately, due to limited access and visibility of the plating site, the surgeon can encounter difficulty screwing the taper threaded, distal end of the drill guide into the polyaxial bushing, especially when the plate is already positioned and/or attached to the bone. This may lead to damage of the bushing threads due to cross threading, which can potentially impair the function of the bushing. Furthermore, the time required to use the drill guide for drilling a hole through each of the many polyaxial bushings may be significant, thereby lengthening the time of the surgical procedure. Moreover, the surgical step of drilling occurs during a crucial phase of the procedure when it is very important to maintain the alignment and reduction of bone fragments while fixation of the fragments is achieved. In addition, extensive fluoroscopy is often used during surgery to direct the bone drill into each of the screw holes to ensure proper alignment. Typically several images are obtained for both the lateral and anterior-posterior views, resulting in significant fluoroscopy time for each hole drilled. Where several holes are drilled, the accumulated time under fluoroscopy and associated radiation exposure can be high.

### SUMMARY OF THE INVENTION

It is therefore an object of the invention to provide an element by which to maneuver the plate during implantation.

It is another object of the invention to provide a guide for a plate which provides increased visibility and access to the plate.

It is also an object of the invention to provide a guide which temporarily converts a large diameter screw hole into a K-wire receiving hole.

It is a further object of the invention to provide a guide for a polyaxial bushing which does not require the step of coupling the guide to the plate during the surgical procedure.

It is an additional object of the invention to provide a guide for a polyaxial bushing which reduces the time of the surgical procedure.

It is still another object of the invention to provide a guide for a polyaxial bushing that minimizes the radiation to which the patient is exposed.

In accord with these objects, which will be discussed in detail below, embodiments of a guide for a fixation plate are provided. Such plate preferably includes screw holes each provided with a polyaxial bushing having an internal triple lead taper thread.

According to a first embodiment of the invention, the guide is a relatively short element including a bottom threaded portion, a top portion, and a central passage for guiding an orthopedic device, e.g., K-wire or drill, into bone. The threaded end is preferably provided with a triple lead taper thread that matches the taper thread of the bushing. The central passage may approximate the minor diameter of threads on a fastener used through the hole to secure the plate to bone, or may be of a smaller diameter for another orthopedic device, e.g., for stably receiving a K-wire. The passage at the top portion is preferably provided with tool engagement structure, e.g., corners for receiving a driver. The short length of the device permits multiple guides to be pre-assembled to the plate at once without unnecessarily obscuring and crowding the area of interest. The guides are coupled to the plate in alignment with the axes of the polyaxial bushings, and such alignment can be set by the manufacturer in accord with preferred preset axes or by a medical professional closer to the time of surgery (before or during) in accord with desired anatomical specifications of a particular procedure.

In a preferred use, the pre-assembled guides are initially lightly screwed into respective polyaxial bushings of the plating system, permitting the surgeon to modify the angle of each guide and bushing to a selected orientation before locking the bushing at the orientation by tightening of the guide into the bushing. This tightening may occur either before or after positioning the plate on the bone. Once the bone holes have been drilled, the surgeon preferably uses a tool to remove each guide before inserting a bone screw into the bushing and drilled hole.

In some embodiments, the top end defines a handle, preferably with external gripping structure, e.g., longitudinal ridges, to facilitate manual manipulation. The guide, preferably pre-assembled to the plate, can be used to maneuver the plate onto the bone.

Also in some embodiments, a guide is provided with an extension. The guide and extension are easily coupled via longitudinal engagement to provide a longer guide for axial alignment, to rotationally insert and remove the guides, and to facilitate certain procedures where a longer guide for orthopedic instrumentation is helpful. The extension can easily be removed from the guide when a short non-obtrusive guide is desired using a quick longitudinal movement.

Additional objects and advantages of the invention will become apparent to those skilled in the art upon reference to the detailed description taken in conjunction with the provided figures.

### BRIEF DESCRIPTION OF THE DRAWINGS

FIG. 1 is a longitudinal section view of a fixation plate provided with a plurality of threaded guides according to the invention;

FIG. 2 is a side elevation view of a first embodiment of a threaded guide according to the invention;

FIG. 3 is a perspective view of the threaded guide of FIG. 2;

FIG. 4 is a perspective view of a fixation plate provided with a second embodiment of a threaded guide according to the invention;

FIG. 5 is a side elevation view of the second embodiment of a threaded guide according to the invention;

FIG. 6 is a perspective view of the threaded guide of FIG. 5;

FIG. 7 is a view similar to FIG. 4 shown with a guide extension coupled to the threaded guide;

FIG. 8 is a side elevation of the guide extension of FIG. 7;

FIG. 9 is a section view along lines 9-9 in FIG. 8;

FIG. 10 is a distal end view of the guide extension of FIG. 7;

FIG. 11 is an exploded side elevation view of a third embodiment of a guide according to the invention;

FIG. 12 is an exploded perspective view of the third embodiment of a guide according to the invention;

FIG. 13 is a broken partial section view of an assembled guide according to the third embodiment of the invention;

FIG. 14 is an exploded side elevation view of a fourth embodiment of a guide according to the invention; and

FIG. 15 is an exploded perspective view of the fourth embodiment of a guide according to the invention.

### DETAILED DESCRIPTION

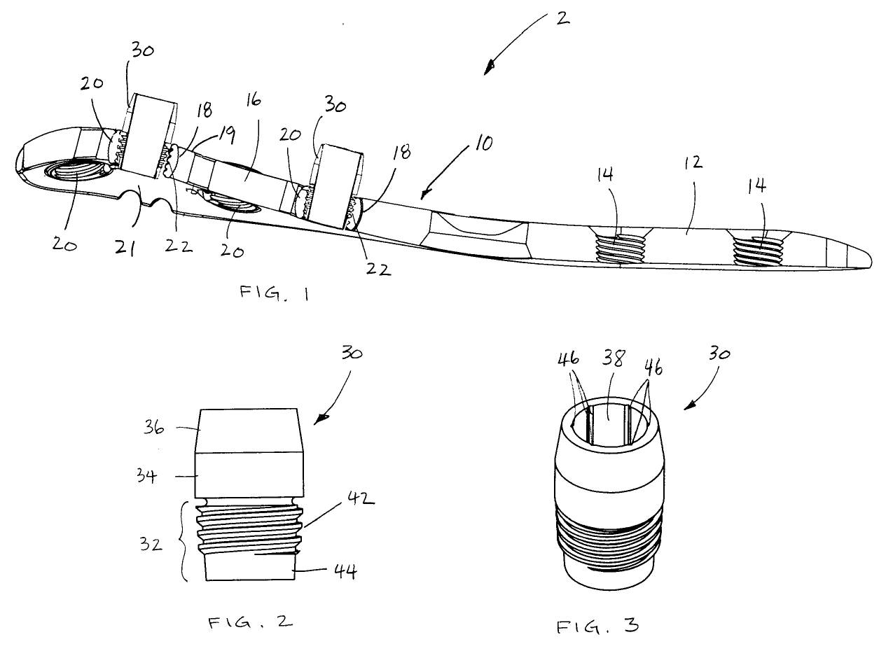

Turning now to FIG. 1, a system 2 including a fixation plate 10 with guides 30 according to the invention is shown. The plate 10 includes a shaft portion 12 including threaded screw holes 14 and a head portion 16 including holes 18. The holes 18 each define a spherically curved inner surface extending between a bone contacting surface 19 and an upper (opposite) surface 21. A polyaxial bushing 20 is provided in each such hole. The interior surface of each bushing 20 includes a triple lead taper thread 22. Such holes and bushings are described in previously incorporated U.S. Pat. No. 5,954,722 to Bono and U.S. Pub. No. 2005/0049594A1 to Wack et al. A removable guide 30 is provided for the bushings 20.

Referring to FIGS. 1 through 3, according to a first embodiment of the invention, the guide 30 is a relatively short device including a bottom portion 32, a central portion 34, a top portion 36, and an axial passage 38. The bottom portion 32 is tapered with triple lead threads 42 sized for engagement with the triple lead taper threads 22 of the polyaxial bushing 20. The triple lead threads 42 preferably extend through approximately 360°. A distalmost end of the bottom portion 32 preferably includes a non-threaded lead-in portion 44 that facilitates insertion of the guide 30 into the bushing 20. The top portion 36 is preferably tapered to increase clearance between other guides 30 coupled to the plate 10. The axial passage 38 in the embodiment shown approximates the minor diameter of a threaded fastener to be used through the bushing 20 and is thus of suitable dimension to receive a drill bit for drilling a hole in the bone for such threaded fastener. Alternatively, the diameter may be sized substantially smaller than the hole defined by bushing 20 for stably receiving a K-wire or other temporary fastener or another orthopedic instrument. The passage 38 at the top end is preferably provided with tool engagement structure, e.g., six corners 46 for receiving a hex driver. Thus, the same tool used to remove the guide (a hex driver) can be used to insert a fastener having a hex slot into the hole thereafter. The short length of the device, preferably not more than one inch, and more preferably approximately 0.35-0.6 inch, permits multiple guides 30 to be assembled to the plate 10 at once without unnecessarily obscuring and crowding the area of interest at the surgical site. The guides 30 are coupled to the plate in alignment with the axes of the polyaxial bushings, and such alignment can be set by the manufacturer in accord with preferred preset axes or by a medical professional closer to the time of surgery (before or during) in accord with desired anatomical specifications of a particular procedure. The guides are not coupled to the plate as tightly as the threaded fastener that will eventually be positioned in its place and tightened within the bushing. Therefore, the guides 30 may be used as small handles to redirect the axial alignment of the bushing 20. As discussed with respect to other embodiments below, guide extensions can be coupled to the guides to facilitate such redirection and the drilling of holes therethrough.

In a preferred use, a plate with guides pre-assembled therein in preferred axial alignments is provided to the surgeon. The pre-assembled guides are preferably lightly screwed into respective polyaxial bushings of the plating system permitting the surgeon to modify the angle of each guide and bushing to a selected orientation (angular displacement) before locking the bushing at the orientation by tightening of the guide into the bushing. This tightening may occur either before or after positioning the plate on the bone. Once the bone holes have been drilled, the surgeon preferably uses a tool, e.g., a hex driver, or a guide extension, discussed below, to remove each guide before inserting a fastener into the bushing and drilled hole.

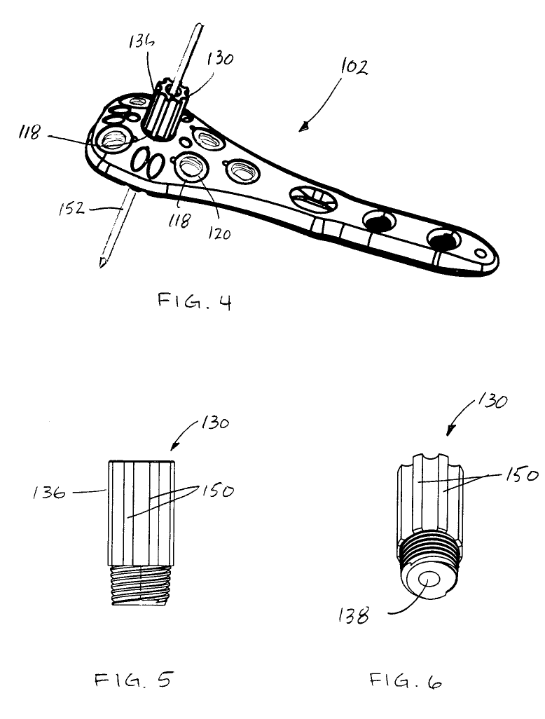

Turning now to FIGS. 4 through 6, a second embodiment of the system 102 is shown. The guide 130 has a top end defining a handle 136, preferably with external gripping structure, e.g., longitudinal ridges 150, to facilitate manual manipulation of the axial orientation of bushing or of the plate as whole. In addition, the passage 138 through the guide in the embodiment shown is sized for a K-wire 152. Thus, a screw hole 118 and bushing 120 that under normal circumstances would be too large for stable fixation by K-wire can be used for temporary fixation of the implant on the bone with K-wire.

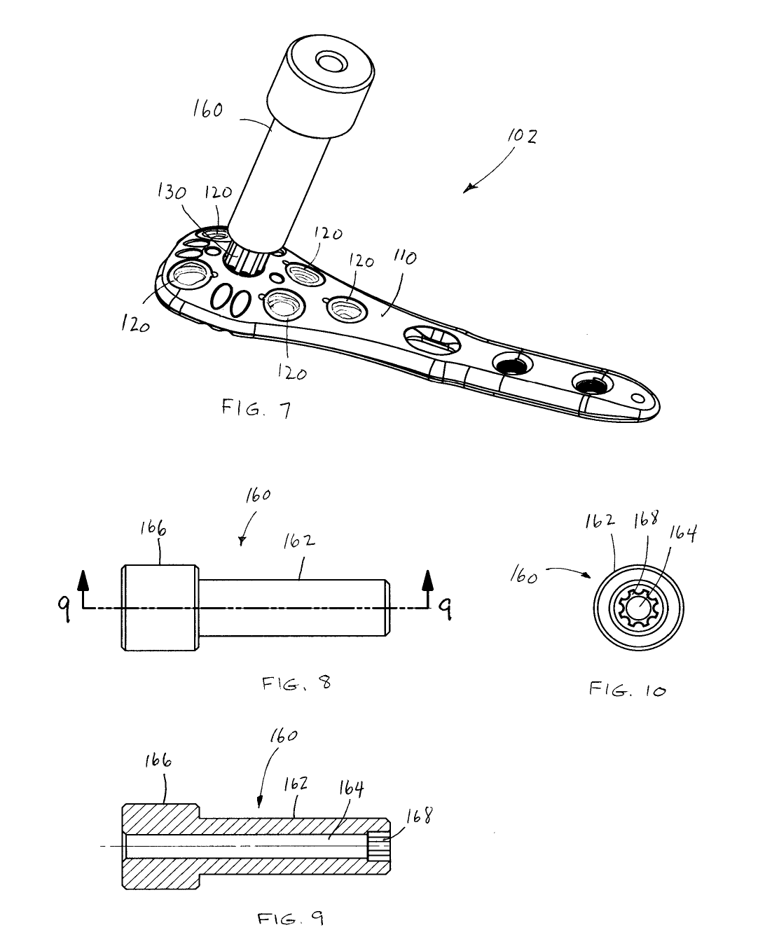

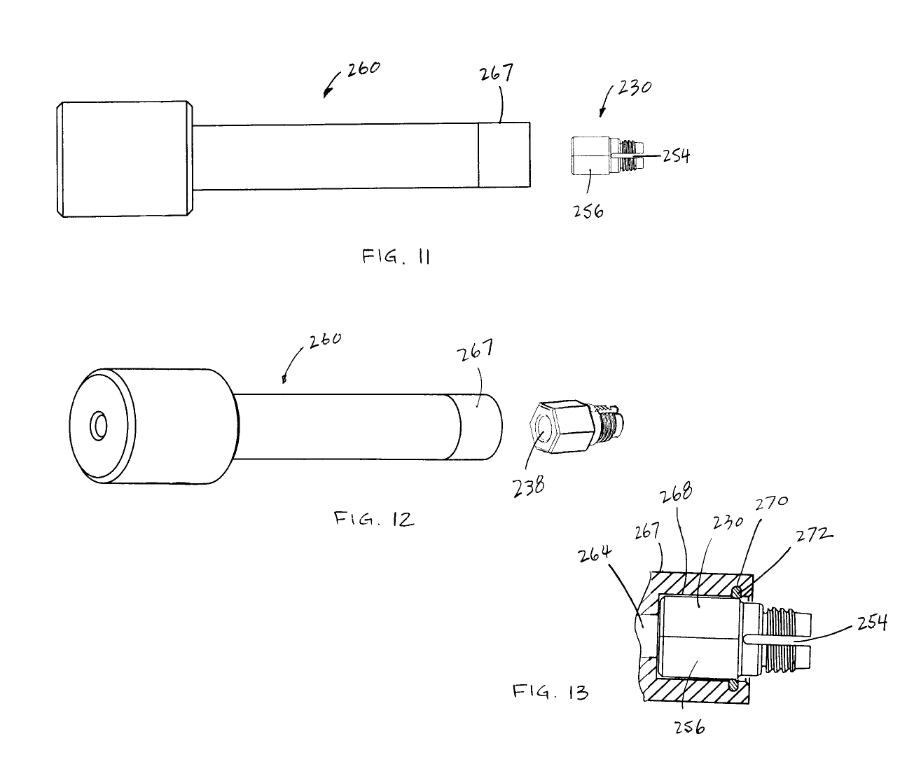

Referring now to FIGS. 7 through 10, the system 102 is shown with a guide extension 160 coupled to the guide 130 in the plate 110. The guide extension 160 includes a shaft 162 defining a central passage 164, a proximal handle 166, and a distal socket 168 for engagement over the top end of the guide 130. When positioned over the guide, the central passages 138, 168 are aligned and preferably of the same dimension so as to provide a composite guiding passageway of longer length for a K-wire or orthopedic device for which the passageway is sized. The guide extension 160 is rotationally fixed relative to the guide 130 so as to also facilitate insertion of the guide 130 into the bushing 120, axial alignment of the guide 130 and bushing 120 relative to the plate 110, tightening of the guide 130 relative to the bushing 120, and removal of the guide 130 from the bushing 120.

Turning now to FIGS. 11 through 13, a third embodiment of a guide 230 and guide extension 260 are shown. The guide 230 is similar to the above in that it is preferably substantially short, provided pre-assembled to the plate, and includes a central passageway for, e.g., K-wire or instrument guidance. The distal portion of the guide 230 includes a diametric slot 254. In the event the guide 230 is inserted with too much force into the bushing 20 (e.g., FIG. 1), the slot 254 allows compression of such distal portion of the guide 230 within the bushing so as to prevent cross threading. The proximal portion of the guide 230 includes a non-circular head 256, preferably hexagonal in shape, for engagement by the extension 260. The distal end 267 of the extension 260 includes socket 268 that captures at least a portion of the proximal portion of the guide 230 in a rotationally interfering manner. Preferably, the distal socket 268 has a corresponding shape, e.g., hex socket, to the proximal portion of the guide. To eliminate play in the assembly of the guide 230 and extension 260, it is preferred to provide a groove 270 in the socket, and a resilient o-ring 272 in the groove that will contact the guide. Once so assembled, the central passages 238, 264 of the guide and extension are aligned.

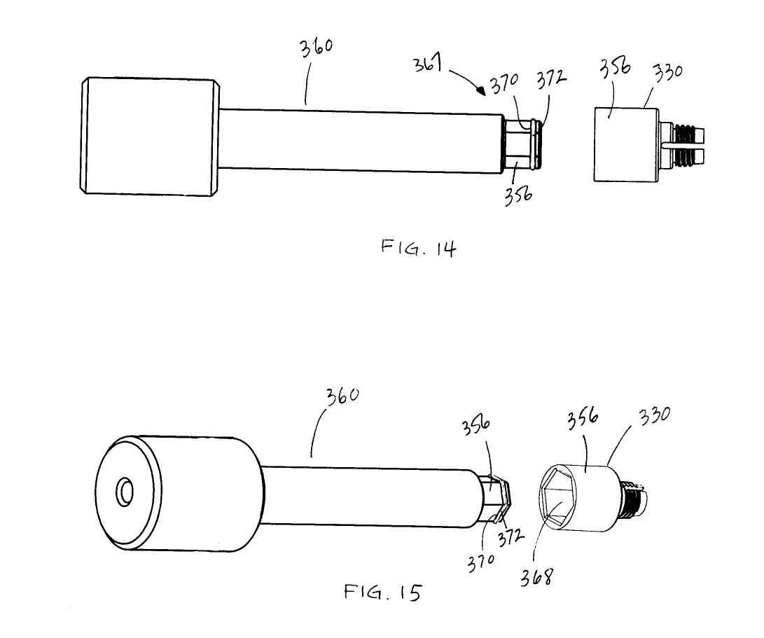

Referring to FIGS. 14 and 15, another embodiment substantially similar to the previous embodiment is shown. The distinguishing features between the embodiments are that a socket 368 is provided to the proximal portion of the guide 330, the distal end 367 of the extension 360 includes a head 356 which engages in a rotationally interfering manner within the socket 368, and a groove 370 and o-ring 372 are provided externally about the head 356 to reduce play in the assembly of the guide and extension.

It is also recognized that the guide extension may be provided with a central passageway defining a diameter that is smaller than the diameter of the central passageway of the guide. Such smaller diameter of the guide extension may, e.g., correspond to the diameter of a K-wire, while the diameter of the guide corresponds to a drill for the locking bone fastener. Using the guide and guide extension, the surgeon can first drill a pilot hole with a K-wire and once satisfied with its location within the bone, then remove the extension and drill through the guide with the regular drill bit for the locking bone fastener.

There have been described and illustrated herein several embodiments of a guide and extension therefor and methods of using the same in conjunction with a plate. While particular embodiments of the invention have been described, it is not intended that the invention be limited thereto, as it is intended that the invention be as broad in scope as the art will allow and that the specification be read likewise. Thus, while particular embodiments have been disclosed, it will be appreciated that the embodiments are exemplary and that the present teaching includes combining features of the various embodiments with each other. Also, while it is recognized that the invention has particular application to humoral plates, more generally the guides and extensions therefor may be applied to any other plate capable of accommodating them, with necessary modifications contemplated. It will therefore be appreciated by those skilled in the art that yet other modifications could be made to the provided invention without deviating from its spirit and scope as claimed.

What is claimed is:

1. An orthopedic fixation plate system, comprising:
a) a fixation plate including a bone contacting surface and an opposite surface, and a hole extending between said surfaces, said hole being spherically curved between said surfaces;
b) a polyaxial bushing provided in said hole;
c) a removable guide coupled in said polyaxial bushing, said guide having a top portion, a bottom portion, a central passageway, and a length of not more than one inch.

2. A system according to claim 1, wherein:
said polyaxial bushing is lockable in a fixed orientation relative to said plate.

3. A system according to claim 2, wherein:
said bushing and said bottom portion of said guide each include tapered threads.

4. A system according to claim 2, wherein:
said bushing and said bottom portion of said guide each include a triple lead thread.

5. A system according to claim 1, wherein:
said guide has a length of 0.35-0.6 inch.

6. A system according to claim 1, wherein:
said plate includes at least two holes, each provided with a polyaxial bushing, and a removable guide being coupled to each polyaxial bushing.

7. A system according to claim 1, wherein:
said bottom portion of said guide includes a distalmost portion that is non-threaded.

8. A system according to claim 1, wherein:
said top portion of said guide is tapered.

9. A system according to claim 1, wherein:
said central passage is substantially smaller than an inner diameter of said bushing.

10. A system according to claim 1, wherein:
said top portion of said guide includes structure that permits rotational interference with said guide by a tool.

11. A system according to claim 1, wherein:
said guide includes external gripping structure to facilitate manipulation.

12. A system according to claim 1, further comprising:
a guide extension that is removably coupled to said guide, said guide extension including a passageway that is aligned with said central passageway of said guide when the guide and guide extension are coupled together, said guide and guide extension being coupled together in a rotationally interfering manner.

13. A system according to claim 12, wherein:
said guide extension includes a resilient member to remove play in the coupling of the guide extension and guide.

14. A system according to claim 12, wherein:
said passageway of said guide extension has a smaller diameter than said central passageway of said guide.

15. An orthopedic plate system for use with a bone fastener with a threaded head for fixation of a bone fracture, comprising:
a) a plate including a bone contacting surface, an opposite surface, and a threaded hole being provided between said surfaces, said hole having a first diameter for receiving the threaded head of the bone fastener for coupling the plate relative to the bone; and
b) a removable guide coupled relative to said plate, said guide having a top portion, a bottom portion including threads which mate with the threaded hole, and a central passageway having a second diameter substantially smaller than said first diameter, said second diameter sized for stably receiving a K-wire substantially smaller in diameter than said threaded head of said bone fastener,
wherein said guide can be removed from said hole prior to inserting the bone fastener into the hole.

16. A system according to claim 15, wherein:
said guide has a length of not more than one inch.

17. A method of fracture fixation, comprising:
a) providing a plate having at least one hole with a polyaxial bushing including an internal thread, and a guide pre-assembled into at least one of said at least one hole;
b) at the option of a medical professional, modifying an axial alignment of at least one bushing by angular displacement of the guide pre-assembled thereto; and
c) temporarily locking an axial alignment for a bushing by rotating the respective guide to tighten the guide into the bushing.

18. A method according to claim 17, wherein:
said providing includes pre-assembling said guide in a predetermined alignment by a manufacturer.

19. A method according to claim 17, wherein:
said providing includes pre-assembling said guide in a predetermined alignment by a medical professional prior to placement of said plate on the bone.

20. A method according to claim 17, wherein:
said rotating is accomplished using a guide extension that rotationally interferes with said guide.

21. A method according to claim 17, wherein:
said modifying an axial alignment is manually performed by direct contact between said guide and a hand.

22. A method according to claim 17, wherein:
said guide has a length of less than one inch.
